(19) United States
(12) Patent Application Publication
(10) Pub. No.: US 2006/0116707 A1
(43) Pub. Date: Jun. 1, 2006

(54) Blood vessel cutter

(75) Inventors: Amir Loshakove, Moshav-Bazra (IL); Ido Kilemnik, Herzelia (IL); Ofer Nativ, Rishon-Lezion (IL)

Correspondence Address: WOLF, BLOCK, SCHORR & SOLIS-COHEN LLP, 250 PARK AVENUE, NEW YORK, NY, 10177, US

(73) Assignee: By-Pass, Inc, Orangeburg (NY)

(21) Appl. No.: 10/528,866

(22) PCT Filed: Sep. 25, 2003
(86) PCT No.: PCT/IL03/00771

§ 371 Date: Mar. 24, 2005

(30) Foreign Application Priority Data
Sep. 25, 2002 (IL) IL02/00790
Aug. 7, 2003 (US) 60492998

(51) Int. Cl. A61B 17/34

(52) U.S. Cl. 606/185

(57) ABSTRACT

A cutter (300) adapted for making an aperture in a blood vessel (810) suitable for receiving an anastomotic connector, the cutter comprising a handle a curved element extending from the handle, a sharp tip (306) at the end of the element, the tip (306) adapted for making at least one entry cut in a blood vessel (810) and at least one exit cut in the same blood vessel a distance from the entry cut while a portion of the curved element is substantially within the vessel lumen and a sharp edge (376) along a portion of the convex aspect of the portion, the edge adapted to cut the vessel as the handle is pulled radially away from the lumen.

### RELATED APPLICATIONS

The present application claims priority from and is a continuation-in-part of PCT application PCT/IL02/00790, filed on Sep. 25, 2002, which designates the US, now published in English as WO 03/026475, the disclosure of which is incorporated herein by reference. It also claims priority as well as the benefit under 119 (e) of U.S. Ser. No. 60/492,998, filed on Aug. 7, 2003. This application is also a continuation-in-part of PCT/IL02/00215, filed on Mar. 18, 2002, PCT/IL01/01019, filed on Nov. 4, 2001, PCT/IL01/00903, filed on Sep. 25, 2001, PCT/IL01/00600, filed on Jun. 28, 2001 and PCT/IL01/00266, filed on Mar. 20, 2001. The disclosure of all of these applications, which designate the US and were filed in English, are incorporated herein by reference.

### FIELD OF THE INVENTION

The present invention relates to devices used during hole-forming in blood vessels.

### BACKGROUND OF THE INVENTION

Making an anastomotic connection between two vessels, for example, a grafted vessel and a coronary artery, bequeaths new life to a heart and the human in which it beats.

To begin this life-saving operation, in a typical implementation, the operator measures the host vessel to determine its diameter and chooses an anastomotic connector of a corresponding size. He cuts a linear aperture along the longitudinal axis of the host vessel, exactly to the size of the connector. The operator then evaluates the incision length to ensure that it indeed has the precise length required. An improper host vessel size evaluation and/or aperture length, can result in fatal consequences, or at least, in some cases, in the need to close up one incision and make another.

Surgery on life sustaining organs, often already weakened by the inadequate blood supply, demands speed to reduce surgical risks. Hence, all the steps required in forming a precision aperture, should occur within a relatively short period of time. Instruments that facilitate precision vessel and aperture evaluation and/or rapid cutting of an aperture and/or simplify the process, can be useful in ensuring a successful anastomotic connection.

### SUMMARY OF THE INVENTION

A broad aspect of some embodiments of the invention relates to ensuring that an aperture formed in a blood vessel is of a desired length. Optionally, a device in accordance with an exemplary embodiment of the invention assists in preventing over cutting of the aperture. Optionally alternatively or additionally, a device in accordance with an exemplary embodiment of the invention is inserted into a blood vessel to begin an incision and may be removed while having caused only minimal damage to the vessel, which does not require stitching shut, for example, only a small puncture wound.

An aspect of some embodiments of the invention relates to a cutter instrument that cuts an aperture in a blood vessel. In an exemplary embodiment, the cutter comprises a post having a curved element with a sharp tip extending from the post, a portion of the convex aspect of the curved element comprising a sharp edge.

In an exemplary embodiment, the tip pierces through a host vessel wall, making an entry puncture. In some embodiments, the tip makes a small cut rather than a puncture. In either case, the tip is optionally designed to prevent tearing and/or forming of a large aperture. The post is manipulated until the tip exits the blood vessel, making an exit puncture at a distance from the entry puncture while a portion of the element moves through the vessel lumen. As only the convex aspect of the cutter is sharp, no damage is caused to the blood vessel beyond these two cuts. In an exemplary embodiment of the invention, the entry manipulation is assisted by the curved element being substantially an arc. By pulling the element radially away from the vessel lumen, the sharp convex edge cuts the portion between the entry and exit cuts, joining the two cuts into a single aperture. Alternatively, if the distance between the entry and exit puncture is not a desired incision length, the cutter may be removed and/or manipulated so that a new exit puncture is made, at a correct distance.

Optionally, markings are provided on the cutter to assist in estimating the expected incision length. For example, the convex edge comprises a first entry marking a first distance from the tip and a second entry marking at a second distance from the tip. In an exemplary embodiment, an entry puncture into the vessel is made with the tip and the element is manipulated in the blood vessel until the entry puncture is aligned with either the first entry marking or the second entry marking.

The post is manipulated until the tip exits the blood vessel, making an exit puncture in the vessel and the cutter is pulled away from the vessel to make an aperture joining the exit puncture with the entry puncture. When the entry puncture has been aligned with the first entry marking, an aperture of a first length results. When the entry puncture has been aligned with the second entry marking, an aperture of a second length results.

In an exemplary embodiment of the invention, the post and the opposing tip set the final length of the incision prior to completing the incision (when only two punctures, easily sealed, e.g., by simple pressure or a dab of adhesive, had been made). Any correction can be made by advancing the post so the opposing tip is retracted into the blood vessel and then repositioning the tip. Alternatively or additionally to setting the length, the post and tip set the exact layout of the incision (e.g., orientation).

In an exemplary embodiment of the invention, the use of a curved cutting edge allows focusing the cutting force on a small part of the vessel, if necessary. Optionally, the use of a curved cutting edge simplifies the control of the position of the cutting edge, by rotating the post. Optionally, the use of a curved cutting edge provides relative motion between the edge and the tissue being cut, as it slides on the edge, possibly assisting in cutting. Optionally, the use of a curved cutting edge focuses the force on a relatively short section of blood vessel wall, when the post is pulled back with greater force.

An aspect of some embodiments of the invention relates to a frame for controlling an incision length. In an exemplary embodiment of the invention, the frame comprises a tip inserted into a blood vessel and a frame section that pinches a section of the blood vessel wall between the tip and the frame, thereby ensuring that cutting (e.g., with a scalpel) can be easily limited to the portion of the blood vessel pinched by the frame and/or bounded by the frame on at least one side.

An aspect of some embodiments of the invention relates to a measurement device, optionally in the form of a slotted elongate ribbon that is used in evaluating the diameter of a host vessel and/or in evaluating an aperture length cut in the vessel. In an exemplary embodiment, the ribbon comprises at least one first slot, having a first opening length, extending across a portion of the ribbon width and at least one first edge having a first dimension adapted for insertion into an aperture in a blood vessel.

In an exemplary embodiment, the at least one first slot is placed around the blood vessel to evaluate whether the vessel is, for example, smaller than the minimal size that can receive an anastomotic connector. When the vessel is deemed to have an appropriate minimal diameter, an aperture length, of an appropriate length for the anastomotic connector, is cut into the host vessel using, for example, the cutter described herein. Following cutting the aperture, the at least one first edge of the elongate ribbon is placed in or near the aperture to determine that the aperture has an appropriate length for the connector.

In an exemplary embodiment, the ribbon comprises multiple slots having different opening dimensions and the vessel is evaluated for reception of two or more anastomotic connectors, each having a different diameter. Alternatively or additionally the ribbon comprises multiple edges having multiple lengths that are adapted to measure two or more aperture lengths in the host vessel.

In an exemplary embodiment of the invention, the measurement device includes a marker for indicating a desired incision length and/or position. In an exemplary embodiment of the invention, the measurement device includes, at one or both of the incision measurement edges, a sponge (or a tissue adhesive) with ink Alternatively or additionally, the measurement device includes a surface suitable for absorbing or accepting ink from a marker and then exuding said ink when in contact with tissue. In use, the marker may be used to indicate a desired incision length prior to cutting, instead of or before testing the incision by inserting the measurement device. Alternatively, the ink marks the blood vessel when the marker is inserted into the incision, thereby marking alongside the incision the actual length of the marker. A cap is optionally provided to protect the marker section of the measurement device, before use.

In an exemplary embodiment, a kit suitable for use during anastomotic surgical procedures comprises at least one blood vessel cutter and at least one blood vessel evaluator.

There is thus provided in accordance with an exemplary embodiment of the invention, apparatus for forming an incision of a controlled size in a blood vessel, comprising:

a sterile penetration tip which is adapted to pierce a wall of the blood vessel; and

an arcuate section having a cutting edge defined on an inner portion thereof, extending from said penetration tip. Optionally, said tip is adapted to pierce said wall without tearing. Alternatively or additionally, said apparatus comprises a handle extending from said arcuate section on an opposite said of said arcuate section.

In an exemplary embodiment of the invention, said arcuate section defines at least one incision length marking thereon. Optionally, said arcuate section defines at least two incision length marking thereon.

In an exemplary embodiment of the invention, said tip, arcuate section and handle lie in a plane.

In an exemplary embodiment of the invention, apparatus includes a cutting edge only on said arcuate section on an inner portion thereof.

In an exemplary embodiment of the invention, a non-cutting section is defined between a forward tip of said penetration tip an said cutting edge, said separation being greater than 0.5 mm.

In an exemplary embodiment of the invention, said cutting edge has a linear extent of less than 20 mm.

In an exemplary embodiment of the invention, said cutting edge has a linear extent of less than 10 mm.

In an exemplary embodiment of the invention, said cutting edge has a linear extent of less than 5 mm.

There is also provided in accordance with an exemplary embodiment of the invention, apparatus for forming an incision of a controlled size in a blood vessel, comprising:

a sterile penetration tip which is adapted to pierce a wall of the blood vessel; and

a body extending from said tip; and

a cutting guide defined on said body. Optionally, said apparatus comprises a frame adapted to lock said wall between said frame and said body, from outside the blood vessel. Alternatively or additionally, said cutting guide comprises a slot sized to receive a cutting blade suitable for cutting blood vessel walls. Optionally, said slot is marked with distance markers. Alternatively or additionally, said slot has a far end at a point less than 20 mm from said penetration tip. Optionally, said slot has a far end at a point less than 10 mm from said penetration tip.

In an exemplary embodiment of the invention, said frame is attached to said body by a hinge. Alternatively or additionally, said frame comprises a cutting stop adjacent said penetration tip and past an end of said cutting guide.

In an exemplary embodiment of the invention, said penetration tip is adapted to pierce said blood vessel without causing a tear.

In an exemplary embodiment of the invention, said body is straight.

There is also provided in accordance with an exemplary embodiment of the invention, a method of forming an incision in a blood vessel, comprising:

(a) inserting a penetration tip into a blood vessel, forming a puncture;

(b) fixing said penetration tip so that it maintains a fixed axial position relative to an axis of the blood vessel; and

(c) cutting a linear aperture guided by an extension of said penetration tip.

Optionally, fixing comprises penetrating said penetration tip out of said blood vessel. Alternatively or additionally, fixing comprises locking said blood vessel to said extension of said tip using a frame on the outside of the blood vessel.

In an exemplary embodiment of the invention, the method comprises determining an expected incision length prior to said cutting. Optionally, the method comprises said fixing if said expected length is not a desired length.

In an exemplary embodiment of the invention, the method comprises removing said penetration tip and repeating (a)-(b) to achieve a desired expected incision length.

In an exemplary embodiment of the invention, comprises comprising retracting said extension to form a cut. Alternatively, cutting comprises guiding a knife along said penetration tip to form a cut.

There is also provided in accordance with an exemplary embodiment of the invention, a method of cutting an incision in a blood vessel, comprising:

(a) inserting a front tip of a sickle shaped cutter into a blood vessel;

(b) manipulating said tip to exit the blood vessel at a different point; and

(c) retracting said sickle shaped cutter to cut the blood vessel. Optionally, the method comprises repositioning said tip prior to said retracting, to exit said blood vessel at a further different point. Alternatively or additionally, said blood vessel is only punctured by said tip and is not damaged in any other way by the sickle cutter, prior to (c).

There is also provided in accordance with an exemplary embodiment of the invention, an evaluator adapted for evaluating a blood vessel, comprising:

a flat elongate element having a width;

at least two slots of different opening sizes extending through a portion of the width and adapted to receive a blood vessel therein;

at least one first edge gauge along the element, the edge gauge having a first dimension,

wherein said first dimension is of an incision length in a side vessel suitable for an end-to-side anastomotic connection using an everted blood vessel having the diameter between the two opening sizes. Optionally, the evaluator includes at least one third slot extending through a portion of the width, the at least one third slot having an opening of a size greater than the other two slots. Optionally, the evaluator includes at least one second gage edge, the edge having a second dimension of an incision length in a side vessel suitable for an end-to-side anastomotic connection using an everted blood vessel having the diameter between the greater size and the two opening sizes.

In an exemplary embodiment of the invention, the evaluator includes a marking section adapted for marking a blood vessel, said marking section being at said end gauge. Optionally, said marking section is at an edge of said end gauge.

There is also provided in accordance with an exemplary embodiment of the invention, a sterilized marking evaluator adapted for evaluating a blood vessel comprising:

a flat elongate element having a width;

at least one first edge gauge along the element, the edge gage having a first dimension; and

a marking section adapted for marking a blood vessel, said marking section being at or near said end gauge. Optionally, said first dimension is smaller than 6 mm.

In an exemplary embodiment of the invention, said marking section is at an edge of said end gauge.

Optionally, said evaluator comprises a cap to protect said marking section when not in use.

In an exemplary embodiment of the invention, said evaluator comprises a second edge gauge with a second dimension and a second marking section thereat.

In an exemplary embodiment of the invention, said marking section is at said end. Alternatively, said marking section is near said end.

There is also provided in accordance with an exemplary embodiment of the invention, a method of cutting an aperture in a blood vessel, comprising:

contacting said vessel with a marker having a fixed marking length; and

cutting along said marking. Optionally, the method comprises measuring a finished aperture with said marker. Alternatively or additionally, the method comprises inking said fixed marking width prior to said contacting.

There is also provided in accordance with an exemplary embodiment of the invention, a method of cutting an aperture in a blood vessel, comprising:

inserting a penetration tip into a blood vessel at a point;

visually identifying on the blood vessel a desired incision, starting at said point; and

cutting according to said visual guiding.

### BRIEF DESCRIPTION OF THE FIGURES

Non-limiting embodiments of the invention will be described with reference to the following description of exemplary embodiments, in conjunction with the figures. The figures are generally not shown to scale and any sizes are only meant to be exemplary and not necessarily limiting. In the figures, identical structures, elements or parts that appear in more than one figure are preferably labeled with a same or similar number in all the figures in which they appear, in which:

FIG. 1 is a side view of a vessel evaluator, in accordance with an exemplary embodiment of the invention;

FIG. 2 is side view of a blood vessel cutter, in accordance with an exemplary embodiment of the invention;

FIGS. 3A-3D are views of the blood vessel cutter of FIG. 2, cutting an aperture in a blood vessel, in accordance with an exemplary embodiment of the invention;

FIG. 4 shows the vessel evaluator of FIG. 1 being used in evaluating an aperture in a blood vessel;

FIG. 5 is a schematic illustration of a marking end of a marking evaluation gauge, in accordance with an exemplary embodiment of the invention; and

FIGS. 6A and 6B illustrate a frame-type incision controller, in accordance with an exemplary embodiment of the invention.

### DETAILED DESCRIPTION OF EXEMPLARY EMBODIMENTS

Blood Vessel Evaluator

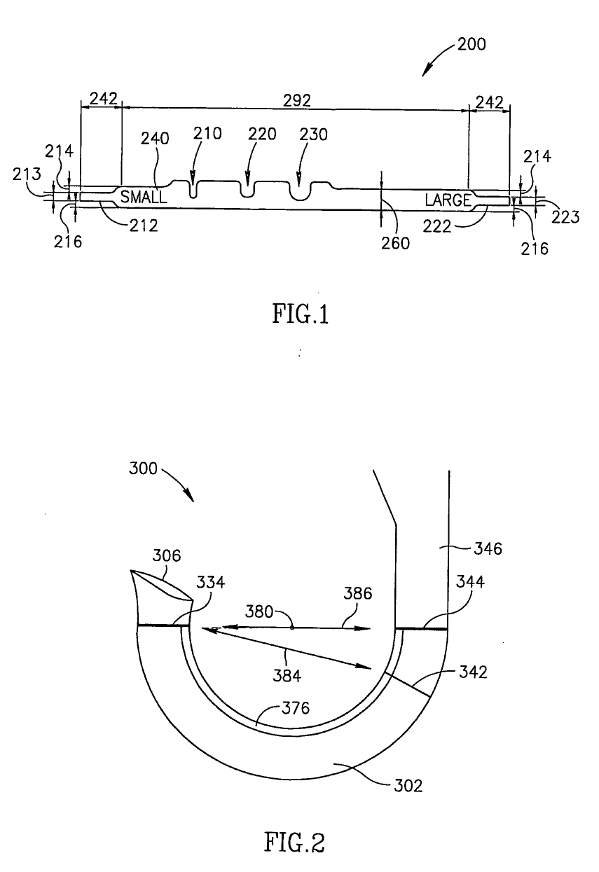

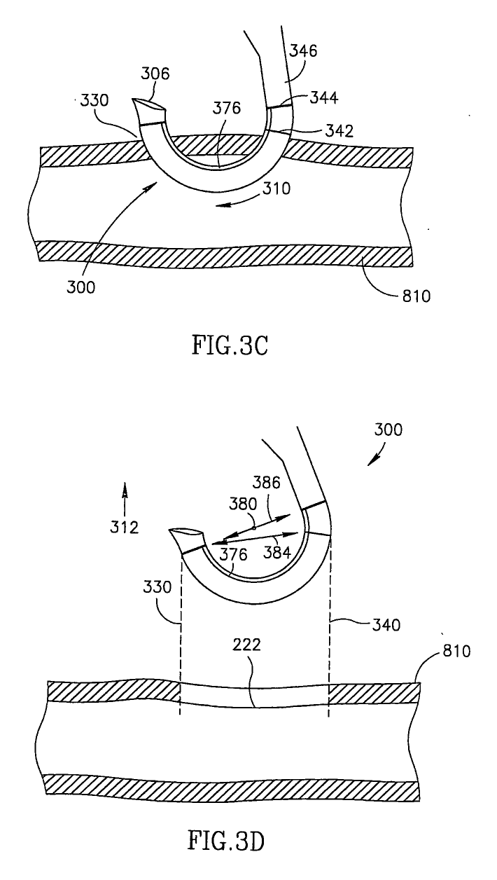

FIG. 1 is an isometric view of a vessel evaluator 200, used in evaluating the diameter of blood vessel 810, (FIG. 3B) in accordance with an exemplary embodiment of the invention. Evaluator 200 comprises notches 210, 220 and 230 that are used to measure the diameter of blood vessel 810 and determine whether a larger or smaller aperture and corresponding anastomotic connector is appropriate.

In an exemplary embodiment, notch 220, having a diameter, for example of 3.5 millimeters, is placed around the blood vessel. If the vessel 810 diameter fits into notch 220, smaller notch 210 is placed around vessel 810. If vessel 810 is larger than smaller notch 210, vessel 810 has a diameter of between 2.0 and 3.5 millimeters, appropriate for an aperture and connector, for example, of 2.5 millimeters.

If, on the other hand, vessel 810 does not fit into notch 220, larger notch 230 is placed around vessel 810. If vessel 810 fits into larger notch 230, it has a diameter between 3.5 and 6.0 millimeters and appropriate for an aperture and connector, for example of 2.9 millimeters. In practice, an anastomosis kit may be provided with only two (or other small number, such as three or four) sizes of connectors, small and large. Size small, uses vessels between the sizes of notches 210 and 220 and size large uses vessels between the sizes of notches 220 and 230. An incision evaluator described below may also be calibrated for these two sizes.

When vessel 810 is larger than larger notch 230 or smaller than smaller notch 210 it is rejected as a graft vessel. In such instances, for example, another graft vessel having an appropriate diameter is sought.

Thus, in an exemplary embodiment of the invention, by using evaluator 200, an operator can rapidly determine the range of the size of vessel 810 and choose an appropriate connector and corresponding incision length.

Blood Vessel Cutter

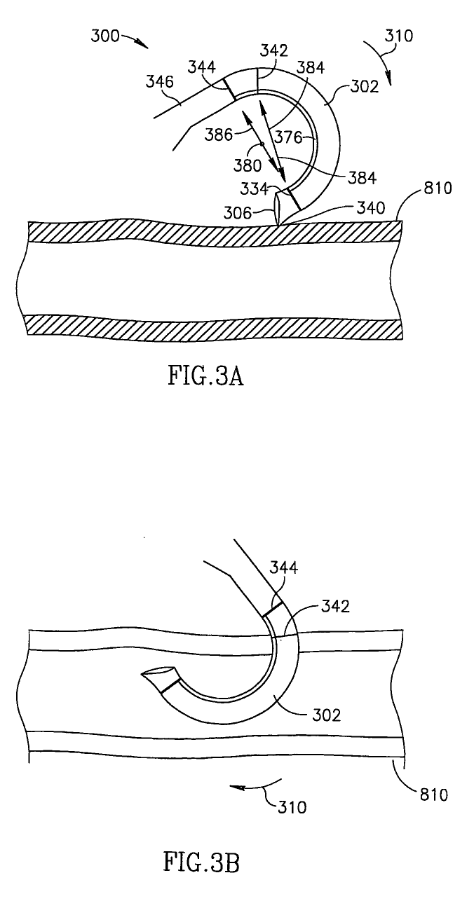

FIG. 2 is a side view of a blood vessel cutter 300 having an overall “J” shape that is appropriate for cutting an incision along a longitudinal axis of vessel 810 (FIG. 3D), in accordance with an exemplary embodiment of the invention. Cutter 300 comprises a post 346 from which a curved blade 302 with a sharp tip 306 project. Typically a portion of curved blade 302 comprises a diameter formed around an origin 380 and a sharp convex edge 376. While a perfect arc is not required, for example a piece-wise section or a non-arc curve may be used, an arc section may have the advantage of easy manipulation as described below.

FIGS. 3A-3D demonstrate operation of cutter 300. Tip 306 is pressed against a surface of blood vessel 810 to make entry cut 340. In an exemplary embodiment of the invention, the cut is actually a small puncture, possibly with no tearing beyond a pinprick. Blade 302 is rotated and/or manipulated, generally in a direction 310. In an exemplary embodiment of the invention, this rotation is assisted by blade 302 being arcuate and not having a cutting edge on its outer curve, as in some embodiments of the invention. FIG. 3C shows tip 306 making an exit cut (or hole) 330. Optionally, an exit marking 334 near tip 306 is provided which can be visualized by the operator. Marking 334 allows the surgeon to determine that tip 306 has fully pierced blood vessel 810 and/or that cutter 300 will not displace inappropriately during cutting.

In FIG. 3D, cutter 300 is then pulled in a direction 312 so that sharp edge 376 cuts the distance between entry cut 340 and exit cut 330.

In an exemplary embodiment, a surgical kit is provided with a first cutter 300 having a first diameter, appropriate for creating an aperture of a first size and a second cutter 300 having a diameter appropriate for creating an aperture of a second size. Alternatively or additionally, a single cutter 300 is provided that can be used to create at least two apertures of different sizes.

In an exemplary embodiment, cutter 300 comprises exit marking 334, a first entry marking 342 a first distance 384 from marking 334 and a second entry marking 344 a second distance 386 from marking 334.

After tip 306 has made entry cut 340 (FIG. 3C), blade 302 is manipulated, for example, so that a first entry marking 342 is aligned with entry cut 340. The operator manipulates tip 306 so it exits vessel 810, making exit cut 330 that he aligns with exit marking 334. When post 346 is pulled in direction 312, aperture 222 of first length 384, for example 2.5 millimeters, is formed.

In an exemplary embodiment, when a larger aperture and connector are required, exit marking 344 is aligned with exit cut 340 and entry cut 330 is aligned with entry marking 344. When post 346 is pulled in direction 312, an aperture having a second length 386, for example 2.9 millimeters, results. In some case, a physician may intentionally stretch or compress the blood vessel, for example during manipulation (FIG. 3B), resulting a differently sized incision. If a physician is found to regularly and unintentionally modify the incision length, a cutter with a suitably changed marking and/or geometry is provided.

Blood Vessel Cutter Specifications

Referring to FIG. 3A, while a radius 386 of the curve of blade 302 is, for example 2.9 millimeters, it could be as small as 2.6 millimeters for smaller host vessels and anastomotic connectors and as large as 3.9 millimeters for larger vessels and larger anastomotic connectors.

While distance 384, including two widths of blade 302, is 2.0 millimeters, it could be as small as 1.4 millimeters, or less, such as 1.2 millimeters for smaller host vessels and anastomotic connectors and as large as 2.9 millimeters for larger vessels and larger anastomotic connectors. The thickness of blade 302, is, for example 0.4 mm and the blade may be smooth or serrated. In an exemplary embodiment of the invention, tip 306 is rounded and needle like, so that it will not cause tears. Rounding can be achieved, for example, using electro-polishing. A section of non-cutting area, for example 0.5 mm, 1 mm or 2 mm is optionally provided between the tip and the cutting area, to prevent inadvertent cutting when tip 306 exits for the vessel wall.

Aperture Evaluator

In an exemplary embodiment, evaluator 200 (FIG. 1) comprises a first end edge 212 having a first measurement 213 of 2.5 millimeters and a second end edge 222 having a second measurement 223 of 2.9 millimeters.

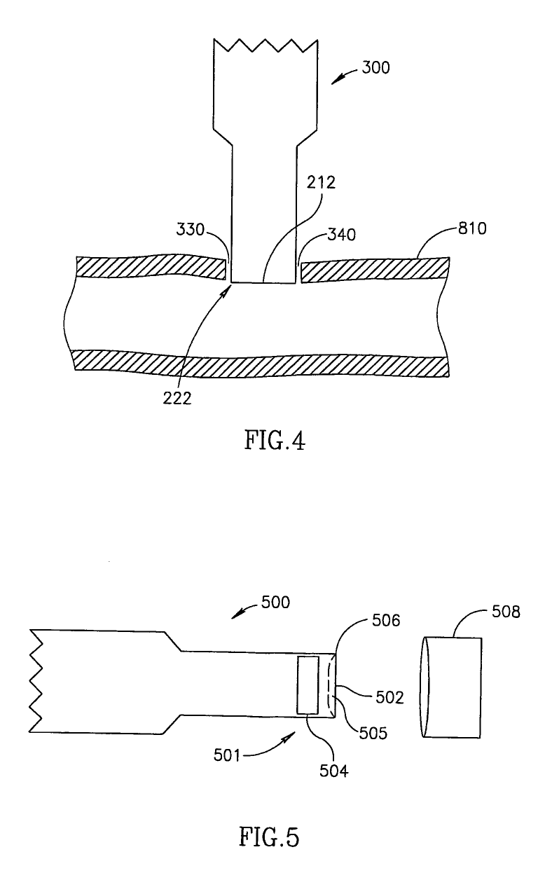

As seen in FIG. 4, end 212 is placed into aperture 222 and a snug fit indicates that aperture 222 is 2.25 millimeters (size “small”). For an aperture of 3 millimeters (size “large”), end 212 is placed into aperture 222 and a snug fit indicates that aperture 222 is of an appropriate length. Thus the operator can proceed with making an anastomotic connection, assured that aperture 222 is of an appropriate length.

If aperture 222 is shorter than necessary, it must be lengthened. If aperture 222 is longer than necessary, it must be closed to forgo, for example, anastomotic connection at this site.

Evaluator Specifications

In an exemplary embodiment, handle 240 has a length 292 of 9 centimeters and a width 260 of 6.0 millimeters. Alternatively or additionally handle 240 could have a length of between 6 and 12 centimeters and width 260 of 4-8 millimeters depending on the vessel being evaluated and/or whether the procedure entails a fully exposed surgical field or a closed surgical field accessed, for example, through a small incision. Additionally, large or smaller lengths are contemplated based upon, for example, the procedure location and the access technique.

A first end 212 and a second end 222 have a projection distance 242 of 1.5 centimeters and offsets 216 and 214 of 2.0 millimeters each. Again, depending upon the size of the vessel being evaluated and/or whether the surgical field is open or closed, these measurements may be varied up or down. Notches 210, 220 and 230 have, for example, a “u” shape, appropriate for receiving blood vessel 810.

In an exemplary embodiment, evaluator 200 and/or cutter 300 comprise materials that are compatible with biologic tissue, for example titanium. Alternatively or additionally all edges are smoothed to prevent trauma to tissue associated with sharp edges.

Marking Evaluator

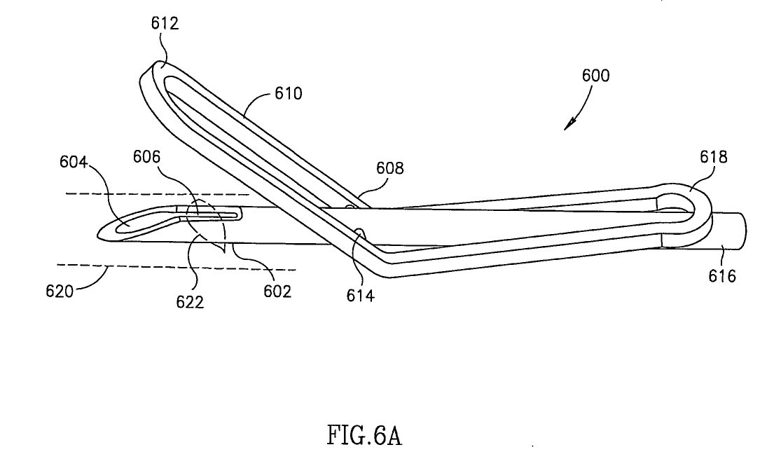

FIG. 5 is a schematic illustration of a marking end 501 of a marking evaluation gauge 500, in accordance with an exemplary embodiment of the invention. A marking area 504 is adapted to apply ink to a wall of vessel 810 (e.g., FIG. 4). In use, once a decision is made to create an incision, marking area 504 is used as a contact stamp to mark the prospective incision with ink. In one embodiment, a marker is used to apply ink to a suitably treated section of gauge 500, so that it will later release the ink. In one embodiment, area 504 is a sponge or adhesive, or is rough, and ink is applied, for example with an ink pad or by running a marker on it. For some markers and/or ink types, no special preparation of area 504 is required. In another embodiment, area 504 already contains the ink and is optionally protected before use by a cap 508. While a solid marking area is shown, in some embodiments, a different pattern is useful, for example, a set of ruled lines.

In an alternative embodiment, area 505 is a marker area, forming a front edge 502 of end 501 and/or on one side thereof. Optionally, area 505 is V shaped, with its apex pointed towards edge 502.

Alternatively or additionally, end 501 is inserted into a formed incision and marking area 504 indicates on the outside of the vessel, the width of end 501, for easier comparison. In an exemplary embodiment of the invention, the forward surface of end 501 is flat edge 502, with sharp corners 506 such that it has a rectangular shape. Alternatively, corners 506 are rounded. Alternatively or additionally, edge 502 is V shaped.

Cutting Frame

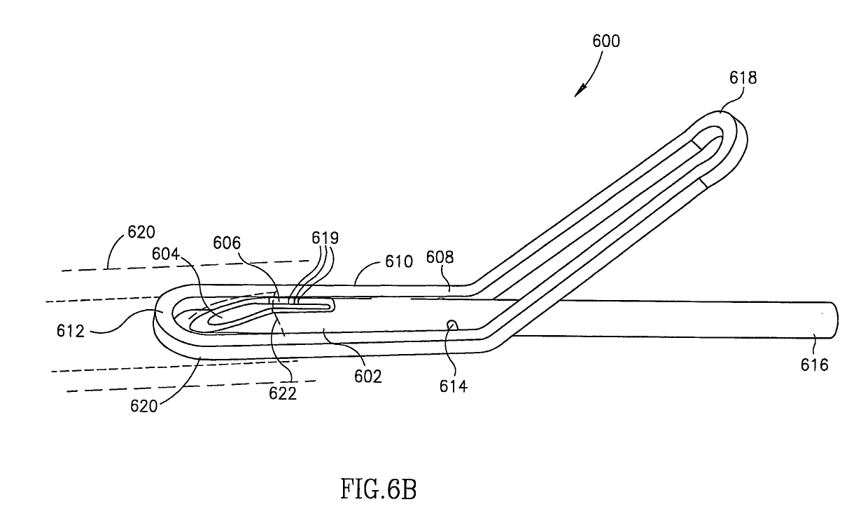

FIGS. 6A and 6B illustrate a cutting frame 600, used for controlling a length of incision in a target vessel 620, in accordance with an exemplary embodiment of the invention.

Frame 600 comprises a penetration section 602 which is inserted into blood vessel 620, forming a puncture therein and having a puncture lip 622. Optionally, penetration section 602 includes a sharp and/or hollow tip 604 to reduce trauma and/or tearing. Optionally, a slot 606 is formed in section 602 and is used for guiding a cutting instrument, as described below. In the example shown, penetration section 602 is a front end of a shaft 616 which can also serve as a handle. Other designs are possible as well.

In addition, cutting frame 600 includes a frame 608 which can be arranged in a desired manner with respect to penetration section 602, as will be described with reference to FIG. 6B. For convenience, in an exemplary embodiment of the invention, frame 608 is attached to penetration section 608 using a hinge 614. A back section 618 of frame 608 is optionally closed to limit rotation of frame 608 in one direction.

In an exemplary embodiment of the invention, cutting frame 600 provides one or both of the following functions: (a) a forward section 612 serves as a stop for a cutting instrument cutting along slot 606; and (b) frame side sections 610, which optionally couple forward section 612 to hinge 608 lock a section of vessel 620, by pinching it between at least a portion of one or both of sides 610 and shaft 616, adjacent penetration tip 602, so that the locked section does not change and the cutting is exact.

As can be seen in FIG. 6B, if a scalpel is used to cut along slot 606 towards forward section 612, the length of the cut is fixed by the distance between lip 622 and forward section 612. Optionally one or more markings 619 are provided adjacent slot 606, so that the final length of an incision can be estimated before cutting is started. Optionally alternatively or additionally, an end of the slot serves as a marking for insertion depth of the penetration tip into the blood vessel optionally, different length slots are provided for different desired lengths. Then, in use, a knife can be inserted in the middle of the slot and used to cut both ways, towards the end of the slot and towards the penetration tip.

If the length is incorrect, frame 608 can be released from penetration section 602 and penetration section 602 advanced into or retracted from vessel 620, as needed, and then frame 608 locked again to penetration section 602, to prevent inadvertent changes in the incision configuration.

In an alternative embodiment of the invention, cutter 300 is used as a cutting guide, by cutting edge 376 being replaced by a slot and a scalpel or other blade guided along the slot. Alternatively, a scalpel can be slid along the body of cutter 300, rather than in a slot.

Alternatively, cutter 300 provides a guillotine cut, in which a cutting blade is guided by handle 346, from outside the blood vessel, towards cutting edge (or slot) 376. Similarly, a cutting edge may be attached to cutting frame 600, to be guided along slot 606.

In some embodiments, one or more of the devices, generally sterilize, described above, are packaged and/or sold with an instruction leaflet, describing the device dimensions and/or situations for which the device should be applied. Also within the scope of the invention are surgical kits comprising sets of medical devices suitable for making anastomotic connections.

It should be appreciated that the above may be varied and still fall within the scope of the invention, for example, by changing the order of steps or by providing embodiments which include features from several described embodiments or by omitting features described herein. Section headings where are provided are intended for aiding navigation and should not be construed to limiting the description to the headings.

Measurements are provided to serve only as exemplary measurements for particular cases. The exact measurements will vary depending on the application. When used in the following claims, the terms “comprises”, “comprising”, “includes”, “including” or the like means “including but not limited to”.

A person skilled in the art will appreciate that the present invention is not limited by what has thus far been described. Rather, the scope of the present invention is limited only by the following claims.

1. Apparatus for forming an incision of a controlled size in a blood vessel, comprising:
a sterile penetration tip which is adapted to pierce a wall of the blood vessel; and
an arcuate section having a cutting edge defined on an inner portion thereof, extending from said penetration tip,
wherein said arcuate section defines at least one incision length marking thereon.

2. Apparatus according to claim 1, wherein said tip is adapted to pierce said wall without tearing.

3. Apparatus according to claim 1, comprising a handle extending from said arcuate section on an opposite end of said arcuate section.

4. Apparatus according to claim 1, wherein said arcuate section defines at least two incision length markings thereon.

5. Apparatus according to claim 1, wherein said arcuate section defines at least two incision length markings, associated with different incision lengths, thereon.

6. Apparatus according to claim 3, wherein said tip, arcuate section and handle lie in a plane.

7. Apparatus according to claim 1, said apparatus includes a cutting edge only on said arcuate section on an inner portion thereof.

8. Apparatus according to claim 1, wherein a non-cutting section is defined between a forward tip of said penetration tip and said cutting edge, said non-cutting section being longer than 0.5 mm.

9. Apparatus according to claim 1, wherein said cutting edge has a linear extent of less than 20 mm.

10. Apparatus according to claim 1, wherein said cutting edge has a linear extent of less than 10 mm.

11. Apparatus according to claim 1, where said cutting edge has a linear extent of less than 5 mm.

12. Apparatus for forming an incision of a controlled size in a blood vessel, comprising:
a sterile penetration tip which is adapted to pierce a wall of the blood vessel;
a body extending from said tip; and
a cutting guide defined an said body.

13. Apparatus according to claim 12, comprising a frame adapted to lock said wall between said frame and said body, from outside the blood vessel.

14. Apparatus according to claim 12, wherein said cutting guide comprises a slot sized to receive a cutting blade suitable for cutting blood vessel walls.

15. Apparatus according to claim 14, wherein said slot is marked with distance markers.

16. Apparatus according to claim 14, wherein said slot has a far end at a point less than 20 mm from said penetration tip.

17. Apparatus according to claim 14, wherein said slot has a far end at a point less than 10 mm from said penetration tip.

18. Apparatus according to claim 13, wherein said frame is attached to said body by a hinge.

19. Apparatus according to claim 13, wherein said frame comprises a cutting stop adjacent said penetration tip and past an end of said cutting guide.

20. Apparatus according to claim 12, wherein said penetration tip is adapted to pierce said blood vessel without causing a tear.

21. Apparatus according to claim 12, wherein said body is straight.

22. A method of forming an incision in a blood vessel, comprising:
(a) inserting a penetration tip into a blood vessel, forming a puncture;
(b) fixing said penetration tip so that it maintains a fixed axial position relative to an axis of the blood vessel; and
(c) cutting a linear aperture guided by an extension of said penetration tip.

23. A method according to claim 22, wherein fixing comprises penetrating said penetration tip out of said blood vessel.

24. A method according to claim 22, wherein fixing comprises locking said blood vessel to said extension of said tip using a frame on the outside of the blood vessel.

25. A method according to claim 22, comprising, determining an expected incision length prior to said cutting.

26. A method according to claim 25, comprising, removing said fixing if said expected length is not a desired length.

27. A method according to claim 22, comprising, removing said penetration tip and repeating (a)-(b) to achieve a desired expected incision length.

28. A method according to claim 22, wherein cutting comprises comprising retracting said extension to form a cut.

29. A method according to claim 22, wherein cutting comprises guiding a knife along said penetration tip to form a cut.

30. A method of cutting an incision in a blood vessel, comprising:
(a) inserting a front tip of a sickle shaped cutter into a blood vessel;
(b) manipulating said tip to exit the blood vessel at a different point;
(c) checking a marking on the cutter to estimate a resulting incision length; and
(d) retracting said sickle shaped cutter to cut the blood vessel, following the checking.

31. A method according to claim 30, comprising repositioning said tip prior to said retracting, to exit said blood vessel at a further different point.

32. A method according to claim 30, wherein said blood vessel is only punctured by said tip and is not damaged in any other way by the sickle cutter, prior to (d).

33. An evaluator adapted for evaluating a blood vessel, comprising:
a flat elongate element having a width;
at least two slots of different opening sizes extending through a portion of the width and adapted to receive a blood vessel therein; and
at least one first edge gauge along the element, the edge gauge having a first dimension,
wherein said first dimension is of an incision length in a side vessel suitable for an end-to-side anastomotic connection using an everted blood vessel having the diameter between the two opening sizes.

34. An evaluator according to claim 33 and including at least one third slot extending through a portion of the width, the at least one third slot having an opening of a size greater than the other two slots.

35. An evaluator according to claim 34 and including at least one second gage edge, the edge having a second dimension of an incision length in a side vessel suitable for an end-to-side anastomotic connection using an everted blood vessel having the diameter between the greater size and the two opening sizes.

36. An evaluator according to claim 33 and including a marking section adapted for marking a blood vessel, said marking section being at said end gauge.

37. An evaluator according to claim 36, wherein said marking section is at an edge of said end gauge.

38. A sterilized marking evaluator adapted for evaluating a blood vessel comprising:
a flat elongate element having a width;
at least one first edge gauge along the element, the edge gauge having a first dimension; and
a marking section adapted for marking a blood vessel, said marking section being at or near said edge gauge.

39. A marking evaluator according to claim 38, wherein said first dimension is smaller than 6 mm.

40. A marking evaluator according to claim 38, wherein said marking section is at an edge of said edge gauge.

41. A marking evaluator according to claim 38, comprising a cap to protect said marking section when not in use.

42. A marking evaluator according to claim 38, comprising a second edge gauge with a second dimension and a second marking section thereat.

43. A marking evaluator according to claim 38, wherein said marking section is near said edge.

44. A method of cutting an aperture in a blood vessel comprising:
contacting said vessel with a marker having a fixed marking length; and
cutting along said marking.

45. A method according to claim 44, comprising measuring a finished aperture with said marker.

46. A method according to claim 44, comprising inking said fixed marking length prior to said contacting.

47. A method of cutting an aperture in a blood vessel, comprising:
inserting a penetration tip into a blood vessel at a point;
visually identifying on the blood vessel a desired incision, starting at said point; and
cutting according to said visual guiding.
